(19) United States
(12) Patent Application Publication
(10) Pub. No.: US 2024/0325753 A1
(43) Pub. Date: Oct. 3, 2024

(54) Applying Tumor Treating Fields (TTFields) to a Body Part in at Least Three Different Directions Using Five Arrays of Electrode Elements

(71) Applicant: Novocure GmbH, Root D4 (CH)

(72) Inventor: Shay LEVI, Haifa (IL)

(73) Assignee: Novocure GmbH, Root D4 (CH)

(21) Appl. No.: 18/617,871

(22) Filed: Mar. 27, 2024

Related U.S. Application Data
(60) Provisional application No. 63/456,041, filed on Mar. 31, 2023.

(51) Int. Cl. A61N 1/36; A61N 1/02; A61N 1/04

(52) U.S. Cl. CPC A61N 1/36034; A61N 1/025; A61N 1/0476; A61N 1/36002

(57) ABSTRACT

Due to the directional nature of tumor treating fields (TTFields), increasing the number of directions at which TTFields are applied can increase the efficacy and volume of the TTFields treatment. The approaches described herein rely on five sets of electrode elements positioned on the subject's body to induce TTFields in three or more directions. The first, second, third, and fourth sets are positioned on the anterior, posterior, right, and left portions of the subject's body, respectively. And the fifth set is positioned on a different posterior location. Treatment proceeds by applying alternating voltages between (a) the anterior set and the posterior set; (b) the left set and the right set; and (c) the fifth set and at least one of the anterior, left, and right sets during successive time slots. This approach can increase the volume that is covered by TTFields and also provide thermal management benefits.

### CROSS REFERENCE TO RELATED APPLICATIONS

This Application claims the benefit of U.S. Provisional Application 63/456,041, filed Mar. 31, 2023, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 50 kHz-1 MHz, 50-500 kHz, 75-300 kHz, or 150-250 kHz). FIG. 1 depicts the prior art Optune® system, which delivers 200 kHz TTFields to patients via four transducer arrays that are placed on the patient's skin near the tumor. The transducer arrays are arranged in two pairs, with one pair of transducer arrays 10L, 10R positioned to the left and right of the tumor, and the other pair of transducer arrays 10A, 10P positioned anterior and posterior to the tumor. Each transducer array is connected via a multi-wire cable to an AC signal generator 15. The AC signal generator (a) sends an AC current through the anterior/posterior (A/P) pair of transducer arrays for 1 second, which induces an electric field with a first direction through the tumor; then (b) sends an AC current through the left/right (L/R) pair of arrays for 1 second, which induces an electric field with a second direction through the tumor; then repeats steps (a) and (b) for the duration of the treatment. In the version of Optune® that is used to treat glioblastoma, each transducer array includes nine electrode elements.

### SUMMARY OF THE INVENTION

One aspect of the invention is directed to a first method of using alternating electric fields to treat a tumor or prevent metastases in a subject's head. The first method comprises positioning a first set of one or more first electrode elements on an anterior surface of the subject's head; positioning a second set of one or more second electrode elements on a posterior surface of the subject's head; positioning a third set of one or more third electrode elements on a left surface of the subject's head; positioning a fourth set of one or more fourth electrode elements on a right surface of the subject's head; and positioning a fifth set of one or more fifth electrode elements on at least one of the subject's neck and the subject's torso. The first method also comprises applying an alternating voltage between the first set and the second set during a plurality of first times within a course of treatment; applying an alternating voltage between the third set and the fourth set during a plurality of second times within the course of treatment; and applying an alternating voltage between the fifth set and at least one of the first, second, third, and fourth sets during a plurality of third times within the course of treatment.

In some instances of the first method, the first times, the second times, and the third times are mutually exclusive, and the first times, the second times, and the third times are interleaved with each other.

In some instances of the first method, the first times, the second times, and the third times are mutually exclusive, and the first times, the second times, and the third times are interleaved with each other in a repeating pattern that repeats at least 100 times.

In some instances of the first method, the fifth set is positioned on a rear portion of the subject's neck, and the step of applying the alternating voltage between the fifth set and at least one of the first, second, third, and fourth sets during the plurality of third times comprises applying the alternating voltage between the fifth set and the first set.

In some instances of the first method, the step of applying the alternating voltage between the fifth set and at least one of the first, second, third, and fourth sets during the plurality of third times comprises (a) applying the alternating voltage between the fifth set and the first set during some of the third times, (b) applying the alternating voltage between the fifth set and the third set during some of the third times, and (c) applying the alternating voltage between the fifth set and the fourth set during some of the third times. Optionally, in these instances, the fifth set may be positioned on a rear portion of the subject's neck.

Another aspect of the invention is directed to a second method of using alternating electric fields to treat a tumor or prevent metastases in a subject's head. The second method comprises applying, during a plurality of first times within a course of treatment, an alternating voltage between a first set of one or more first electrode elements positioned on an anterior surface of the subject's head and a second set of one or more second electrode elements positioned on a posterior surface of the subject's head. The second method also comprises applying, during a plurality of second times within the course of treatment, an alternating voltage between a third set of one or more third electrode elements positioned on a left surface of the subject's head and a fourth set of one or more fourth electrode elements positioned on a right surface of the subject's head. And the second method also comprises applying, during a plurality of third times within the course of treatment, an alternating voltage between a fifth set of one or more fifth electrode elements positioned on at least one of the subject's neck and the subject's torso and at least one of the first, second, third, and fourth sets.

Some instances of the second method further comprise positioning the first set on the anterior surface; positioning the second set on the posterior surface; positioning the third set on the left surface; positioning the fourth set on the right surface; and positioning the fifth set on at least one of the subject's neck and the subject's torso.

In some instances of the second method, the first times, the second times, and the third times are mutually exclusive, and the first times, the second times, and the third times are interleaved with each other.

In some instances of the second method, the first times, the second times, and the third times are mutually exclusive, and the first times, the second times, and the third times are interleaved with each other in a repeating pattern that repeats at least 100 times.

In some instances of the second method, the fifth set is positioned on a rear portion of the subject's neck, and the step of applying the alternating voltage between the fifth set and at least one of the first, second, third, and fourth sets during the plurality of third times comprises applying the alternating voltage between the fifth set and the first set.

In some instances of the second method, the step of applying the alternating voltage between the fifth set and at least one of the first, second, third, and fourth sets during the plurality of third times comprises (a) applying the alternating voltage between the fifth set and the first set during some of the third times, (b) applying the alternating voltage between the fifth set and the third set during some of the third times, and (c) applying the alternating voltage between the fifth set and the fourth set during some of the third times. In these instances, the fifth set may optionally be positioned on a rear portion of the subject's neck, on a front portion of the subject's torso, or on both a rear portion of the subject's neck and a front portion of the subject's torso.

Another aspect of the invention is directed to a first apparatus comprising a signal generator, a plurality of switches, and a controller. The signal generator is configured to generate an alternating output voltage. The plurality of switches is configured to, depending on a state of at least one control input, either (a) operate in a first state in which the alternating output voltage is routed to appear across a first output and a second output, (b) operate in a second state in which the alternating output voltage is routed to appear across a third output and a fourth output, or (c) operate in a third state in which the alternating output voltage is routed to appear across a fifth output and at least one of the first, second, third, and fourth outputs. And the controller is configured to send control signals to the at least one control input, wherein the control signals command the plurality of switches to operate in the first state during a plurality of first times within a course of treatment, operate in the second state during a plurality of second times within the course of treatment, and operate in the third state during a plurality of third times within the course of treatment.

In some embodiments of the first apparatus, the controller is configured such that the first times, the second times, and the third times are mutually exclusive, and such that the first times, the second times, and the third times are interleaved with each other.

In some embodiments of the first apparatus, the controller is configured such that the first times, the second times, and the third times are mutually exclusive, and such that the first times, the second times, and the third times are interleaved with each other in a repeating pattern that repeats at least 100 times.

In some embodiments of the first apparatus, the plurality of switches is configured to, while operating in the third state, route the alternating output voltage to appear across the fifth output and the first output.

In some embodiments of the first apparatus, the controller and the plurality of switches are configured such that: during some of the third times, the plurality of switches routes the alternating output voltage to appear across the fifth output and the first output, during some of the third times, the plurality of switches routes the alternating output voltage to appear across the fifth output and the third output, and during some of the third times, the plurality of switches routes the alternating output voltage to appear across the fifth output and the fourth output.

Some embodiments of the first apparatus further comprise a first set of one or more first electrode elements positioned on an anterior surface of a subject's head and wired to the first output; a second set of one or more second electrode elements positioned on a posterior surface of the subject's head and wired to the second output; a third set of one or more third electrode elements positioned on a left surface of the subject's head and wired to the third output; a fourth set of one or more fourth electrode elements positioned on a right surface of the subject's head and wired to the fourth output; and a fifth set of one or more fifth electrode elements positioned on at least one of the subject's neck and the subject's torso and wired to the fifth output.

Another aspect of the invention is directed to a third method of using alternating electric fields to treat a tumor or prevent metastases in a subject's body. The third method comprises positioning a first set of one or more first electrode elements on a first anterior surface of the subject's body; positioning a second set of one or more second electrode elements on a first posterior surface of the subject's body; positioning a third set of one or more third electrode elements on a left surface of the subject's body; positioning a fourth set of one or more fourth electrode elements on a right surface of the subject's body; and positioning a fifth set of one or more fifth electrode elements on at least one of a second anterior surface of the subject's body and a second posterior surface of the subject's body. The third method also comprises applying an alternating voltage between the first set and the second set during a plurality of first times within a course of treatment; applying an alternating voltage between the third set and the fourth set during a plurality of second times within the course of treatment; and applying an alternating voltage between the fifth set and at least one of the first, second, third, and fourth sets during a plurality of third times within the course of treatment.

In some instances of the third method, the first times, the second times, and the third times are mutually exclusive, and the first times, the second times, and the third times are interleaved with each other. In some instances of the third method, the first times, the second times, and the third times are mutually exclusive; and the first times, the second times, and the third times are interleaved with each other in a repeating pattern that repeats at least 100 times.

In some instances of the third method, the fifth set is positioned on the second posterior surface of the subject's body. In these instances, the step of applying the alternating voltage between the fifth set and at least one of the first, second, third, and fourth sets during the plurality of third times comprises applying the alternating voltage between the fifth set and the first set.

In some instances of the third method, the step of applying the alternating voltage between the fifth set and at least one of the first, second, third, and fourth sets during the plurality of third times comprises (a) applying the alternating voltage between the fifth set and the first set during some of the third times, (b) applying the alternating voltage between the fifth set and the third set during some of the third times, and (c) applying the alternating voltage between the fifth set and the fourth set during some of the third times. Optionally, in these instances, the fifth set is positioned on the second posterior surface of the subject's body.

Another aspect of the invention is directed to a fourth method of using alternating electric fields to treat a tumor or prevent metastases in a subject's body. The fourth method comprises applying, during a plurality of first times within a course of treatment, an alternating voltage between a first set of one or more first electrode elements positioned on a first anterior surface of the subject's body and a second set of one or more second electrode elements positioned on a first posterior surface of the subject's body. The fourth method also comprises applying, during a plurality of second times within the course of treatment, an alternating voltage between a third set of one or more third electrode elements positioned on a left surface of the subject's body and a fourth set of one or more fourth electrode elements positioned on a right surface of the subject's body. And the fourth method also comprises applying, during a plurality of third times within the course of treatment, an alternating voltage between a fifth set of one or more fifth electrode elements positioned on at least one a second anterior surface of the subject's body and a second posterior surface of the subject's body and at least one of the first, second, third, and fourth sets.

Some instances of the fourth method further comprise positioning the first set on the first anterior surface; positioning the second set on the first posterior surface; positioning the third set on the left surface; positioning the fourth set on the right surface; and positioning the fifth set on at least one of the second anterior surface of the subject's body and the second posterior surface of the subject's body.

In some instances of the fourth method, the first times, the second times, and the third times are mutually exclusive; and the first times, the second times, and the third times are interleaved with each other. In some instances of the fourth method, the first times, the second times, and the third times are mutually exclusive; and the first times, the second times, and the third times are interleaved with each other in a repeating pattern that repeats at least 100 times.

In some instances of the fourth method, the fifth set is positioned on the second posterior surface of the subject's body; and the step of applying the alternating voltage between the fifth set and at least one of the first, second, third, and fourth sets during the plurality of third times comprises applying the alternating voltage between the fifth set and the first set.

In some instances of the fourth method, the step of applying the alternating voltage between the fifth set and at least one of the first, second, third, and fourth sets during the plurality of third times comprises (a) applying the alternating voltage between the fifth set and the first set during some of the third times, (b) applying the alternating voltage between the fifth set and the third set during some of the third times, and (c) applying the alternating voltage between the fifth set and the fourth set during some of the third times. In these instances, the fifth set may optionally be positioned on the second posterior surface of the subject's body, or on both a rear portion of the subject's neck and a front portion of the subject's torso.

Another aspect of the invention is directed to a second apparatus. The second apparatus comprises a signal generator, a plurality of switches, and a controller. The signal generator is configured to generate an alternating output voltage. The plurality of switches is configured to, depending on a state of at least one control input, either (a) operate in a first state in which the alternating output voltage is routed to appear across a first output and a second output, (b) operate in a second state in which the alternating output voltage is routed to appear across a third output and a fourth output, or (c) operate in a third state in which the alternating output voltage is routed to appear across a fifth output and at least one of the first, second, third, and fourth outputs. And the controller is configured to send control signals to the at least one control input, wherein the control signals command the plurality of switches to operate in the first state during a plurality of first times within a course of treatment, operate in the second state during a plurality of second times within the course of treatment, and operate in the third state during a plurality of third times within the course of treatment.

In some embodiments of the second apparatus, the controller is configured such that the first times, the second times, and the third times are mutually exclusive, and such that the first times, the second times, and the third times are interleaved with each other. In some embodiments of the second apparatus, the controller is configured such that the first times, the second times, and the third times are mutually exclusive, and such that the first times, the second times, and the third times are interleaved with each other in a repeating pattern that repeats at least 100 times. In some embodiments of the second apparatus, the plurality of switches is configured to, while operating in the third state, route the alternating output voltage to appear across the fifth output and the first output.

In some embodiments of the second apparatus, the controller and the plurality of switches are configured such that: during some of the third times, the plurality of switches routes the alternating output voltage to appear across the fifth output and the first output; during some of the third times, the plurality of switches routes the alternating output voltage to appear across the fifth output and the third output; and during some of the third times, the plurality of switches routes the alternating output voltage to appear across the fifth output and the fourth output.

Some embodiments of the second apparatus further comprise a first set of one or more first electrode elements positioned on a first anterior surface of a subject's body and wired to the first output; a second set of one or more second electrode elements positioned on a first posterior surface of the subject's body and wired to the second output; a third set of one or more third electrode elements positioned on a left surface of the subject's body and wired to the third output; a fourth set of one or more fourth electrode elements positioned on a right surface of the subject's body and wired to the fourth output; and a fifth set of one or more fifth electrode elements positioned on at least one of a second anterior surface of the subject's body and a second posterior surface of the subject's body, and wired to the fifth output.

### BRIEF DESCRIPTION OF THE DRAWINGS

FIG. 1 depicts the prior art Optune® system, which delivers 200 kHz TTFields to patients using four transducer arrays.

FIG. 2 depicts an embodiment in which four sets of electrode elements are positioned on the subject's head, and a fifth set of electrode elements is positioned on the subject's neck.

FIG. 3A depicts one example of a timing diagram for energizing the sets of electrode elements shown in FIG. 2 that will apply TTFields to the subject's brain in additional directions.

FIG. 3B depicts another example of a timing diagram for energizing the sets of electrode elements shown in FIG. 2 that will apply TTFields to the subject's brain in additional directions.

FIG. 4 depicts one example of a hardware block diagram that can be used to apply the voltages to the sets of electrode elements shown in FIG. 2.

FIG. 5 depicts another example of a timing diagram for energizing the sets of electrode elements shown in FIG. 2 that will apply TTFields to the subject's brain in additional directions.

FIG. 6 depicts another embodiment in which four sets of electrode elements are positioned on the subject's head, and a fifth set of electrode elements is positioned on the subject's torso.

FIG. 7 depicts another embodiment in which five sets of electrode elements are positioned about a body part.

FIG. 8 depicts another embodiment in which five sets of electrode elements are positioned about a body part.

Various embodiments are described in detail below with reference to the accompanying drawings, wherein like reference numerals represent like elements.

### DESCRIPTION OF THE PREFERRED EMBODIMENTS

TTFields are most effective when the lines of force of the electric field are oriented generally parallel to the long axis of the hourglass-shaped cell during mitosis. And applying TTFields in different directions at different times increases the overall effectiveness of the treatment, because the field orientation that is most effective will be applied to a larger population of the tumor cells as they divide. In the prior art Optune® system, the direction of the TTFields switches back and forth between two different directions, and each of these two directions relies on a pair of transducer arrays (with the two members of any given pair positioned on opposite sides of the subject's head).

This application describes a variety of approaches for increasing the effectiveness of TTFields by increasing the number of directions at which TTFields are applied. The approaches described herein can also be used to increase the effectiveness of TTFields by increasing the overall volume that is covered by the TTFields (which can be advantageous when the body part being treated contains multiple regions of interest or a single region of interest that occupies a large volume within the subject's body), and/or to provide thermal management benefits (e.g., by reducing the duty cycle of transducer arrays that may be approaching their temperature limit, which can increase the amount of time that TTFields can be applied).

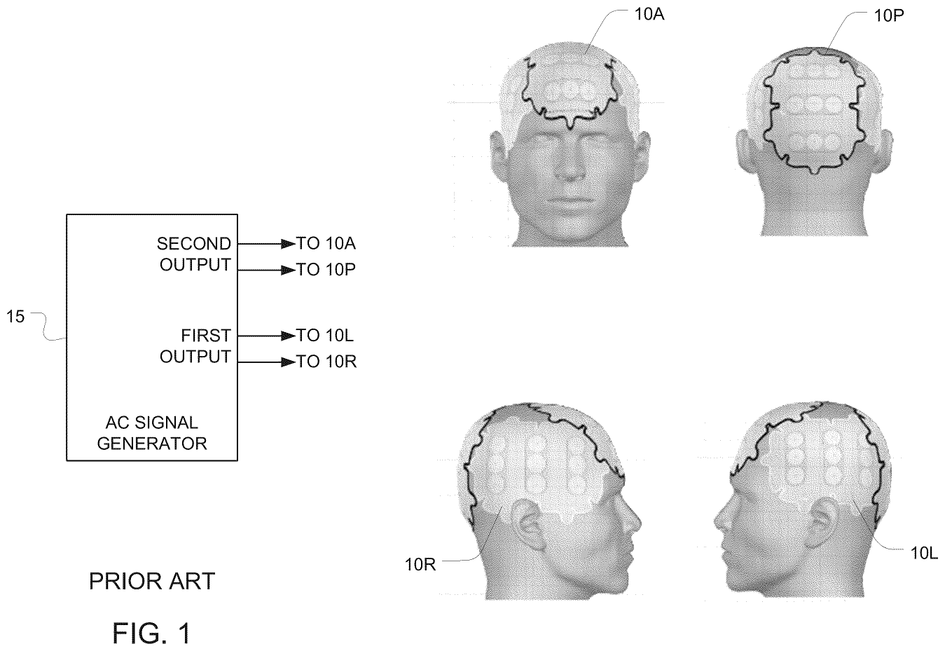

A first approach for increasing the effectiveness of TTFields relies on an additional pair of transducer arrays (i.e., in addition to the anterior/posterior and left/right transducer arrays described above in connection with Optune® and depicted in FIG. 1). Each of these transducer arrays includes a set of one or more electrode elements (e.g., between 1 and 30 electrode elements). The additional pair of transducer arrays is used to induce a roughly vertical electric field through the subject's head. More specifically, one member of the additional pair is positioned on the crown of the subject's head, and the other member of the additional pair is positioned on the subject's neck or upper torso. To use this approach, the system (a) energizes the anterior/posterior pair for a period of time (e.g., 1 s), which induces an electric field with a first direction through the tumor, then (b) energizes the left/right pair for a period of time, which induces an electric field with a second direction through the tumor, then (c) energizes the top/bottom pair for a period of time, which induces an electric field with a third direction through the tumor; and then repeats this three-step sequence for the duration of the treatment. Notably, by adding the top/bottom pair of transducer arrays, a third generally vertical direction is added to the original two generally horizontal directions.

But implementing this first approach can be difficult because, as seen in FIG. 1, the anterior, posterior, left, and right transducer arrays 10A/P/L/R take up so much space that very little room is left to position an additional transducer array on the crown of the subject's head. And while this difficulty can be addressed by reducing the size of the transducer arrays 10A/P/L/R to make room for the additional transducer array, reducing the size of the transducer arrays introduces two problems. First, it reduces the volume within the subject's brain that will be covered by the generally horizontal TTFields created using the original transducer arrays 10A/P/L/R. And second, due to thermal considerations, smaller transducer arrays typically have to operate at lower currents to prevent overheating. And because lower currents will reduce the intensity of the electric field (which will in turn reduce the efficacy of the treatment), operating at lower currents is not desirable.

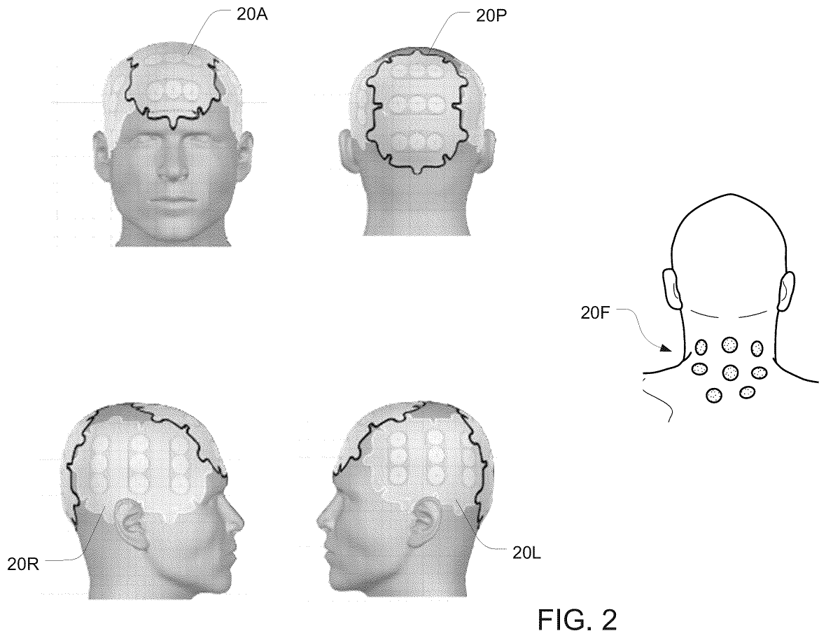

FIGS. 2-5 depict a second approach for increasing the effectiveness of TTFields by increasing the number of directions at which the TTFields are applied. In this approach, as best seen in FIG. 2, a first set of one or more first electrode elements 20A is positioned on an anterior surface of the subject's head; a second set of one or more second electrode elements 20P is positioned on a posterior surface of the subject's head; a third set of one or more third electrode elements 20L is positioned on a left surface of the subject's head; and a fourth set of one or more fourth electrode elements 20R is positioned on a right surface of the subject's head. In addition, a fifth set of one or more fifth electrode elements 20F is positioned on the subject's neck (e.g., on the back of the subject's neck as depicted in FIG. 2).

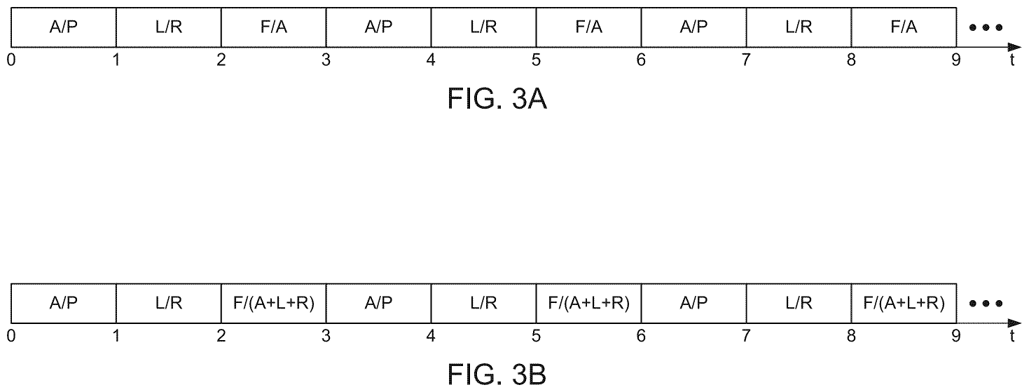

FIG. 3A depicts one example of a timing diagram for energizing the various sets of one or more electrode elements 20A, 20P, 20L, 20R, and 20F that will apply TTFields to the subject's brain in more than two directions. During the first second of operation (i.e., between t=0 and t=1), an alternating voltage is applied between the anterior and posterior sets 20A, 20P, which will induce an electric field in the subject's brain with a general direction that runs from front to back. During the second of operation (i.e., between t=land t=2), an alternating voltage is applied between the left and right sets 20L, 20R, which will induce an electric field in the subject's brain with a general direction that runs from left to right. And during the third second of operation (i.e., between t=2 and t=3), an alternating voltage is applied between the fifth set of electrode elements 20F and the anterior set of electrode elements 20A, which will induce an electric field in the subject's brain in a direction that has a vertical component. This three-part pattern then repeats (e.g., at least 100 times) for the duration of the course of treatment (i.e., from t=3 onward). Note that while each interval of time is 1 second long in the example above, shorter or longer durations (e.g., from 0.1 s to 100 s) can also be used. Note also that in the example above, the anterior/posterior, left/right, and fifth/anterior timeslots are interleaved with each other and are mutually exclusive.

Notably, this approach applies TTFields to the subject's brain in three directions without reducing the size of the transducer arrays that are positioned on the anterior, posterior, left, and right surfaces of the subject's head. And due to the directional nature of TTFields, adding the new direction with the vertical component to the two original generally horizontal directions can advantageously increase the overall effectiveness of the treatment. This approach also increases the overall volume that is covered by the TTFields (which can be advantageous when the body part being treated contains multiple regions of interest or a single region of interest that occupies a large volume within the subject's body).

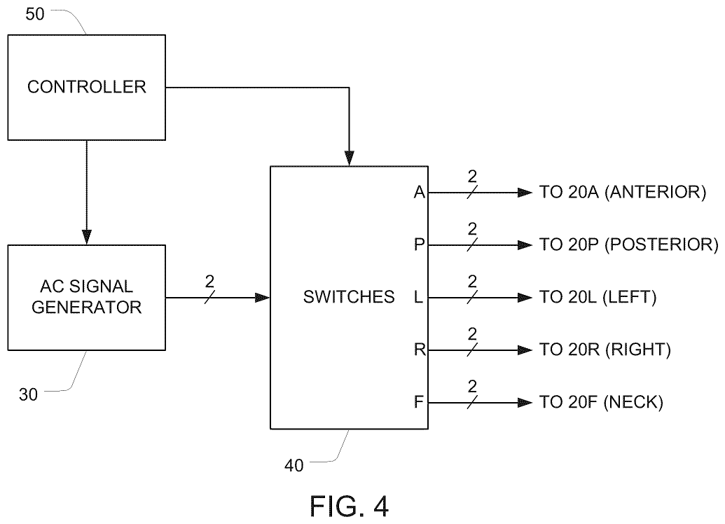

FIG. 4 depicts one example of a hardware block diagram that can be used to apply the voltages to the various sets of one or more electrode elements 20A, 20P, 20L, 20R, and 20F as described above in connection with FIG. 3A. In this example, the AC signal generator 30 generates an AC output voltage at a frequency between 50 kHz and 1 MHz (e.g., 50 kHz-1 MHz, 50-500 kHz, 75-300 kHz, or 150-250 kHz). A bank of electronic switches 40 (which may be implemented e.g., using a set of FETs) is configured to route the output of the AC signal generator to either (1) appear across terminals A and P, (2) appear across terminals L and R, or (3) appear across terminals F and A, depending on the state of a control signal that arrives from the controller 50. The controller 50 is programmed to generate the A/P, L/R, and F/A control signals illustrated in FIG. 3A in the illustrated sequence. These control signals command the switches 40 to operate in the first state (i.e., the A/P state) during a plurality of first times within a course of treatment, operate in the second state (i.e., the L/R state) during a plurality of second times within the course of treatment, and operate in the third state (i.e., the F/A state) during a plurality of third times within the course of treatment.

FIG. 3B depicts another example of a timing diagram for energizing the various sets of one or more electrode elements 20A, 20P, 20L, 20R, and 20F that will apply TTFields to the subject's brain in more than two directions. During the first second of operation (i.e., between t=0 and t=1), an alternating voltage is applied between the anterior and posterior sets 20A, 20P, which will induce an electric field in the subject's brain with a general direction that runs from front to back. During the second of operation (i.e., between t=land t=2), an alternating voltage is applied between the left and right sets 20L, 20R, which will induce an electric field in the subject's brain with a general direction that runs from left to right. And during the third second of operation (i.e., between t=2 and t=3), an alternating voltage is applied between (i) the fifth set of electrode elements 20F and (ii) the anterior, left, and right sets of electrode elements 20A, 20L, 20R, which will induce an electric field in the subject's brain in a direction that has a vertical component. This three-part pattern then repeats (e.g., at least 100 times) for the duration of the course of treatment (i.e., from t=3 onward). Note that while each interval of time is 1 second long in the example above, shorter or longer durations (e.g., from 0.1 s to 100 s) can also be used. Note also that in the example above, the anterior/posterior, left/right, and fifth/(anterior+left+right) timeslots are interleaved with each other and are mutually exclusive.

Notably, this approach applies TTFields to the subject's brain in three directions without reducing the size of the transducer arrays that are positioned on the anterior, posterior, left, and right surfaces of the subject's head. And due to the directional nature of TTFields, adding the new direction with the vertical component to the two original generally horizontal directions can advantageously increase the overall effectiveness of the treatment. This approach also increases the overall volume that is covered by the TTFields (which can be advantageous when the body part being treated contains multiple regions of interest or a single region of interest that occupies a large volume within the subject's body), and/or to provide thermal management benefits (e.g., by reducing the duty cycle of transducer arrays that may be approaching their temperature limit, which can increase the amount of time that TTFields can be applied).

The same hardware described above in connection with FIG. 4 can be used to implement the sequence depicted in FIG. 3B. In this example, the AC signal generator 30 generates an AC output voltage at a frequency between 50 kHz and 1 MHz (e.g., 50 kHz-1 MHz, 50-500 kHz, 75-300 kHz, or 150-250 kHz). The bank of electronic switches 40 (which may be implemented e.g., using a set of FETs) is configured to route the output of the AC signal generator to either (1) appear across terminals A and P, (2) appear across terminals L and R, or (3) appear across terminals F and (A and L and R), depending on the state of a control signal that arrives from the controller 50. The controller 50 is programmed to generate the A/P, L/R, and F/(A+L+R) control signals illustrated in FIG. 3B in the illustrated sequence. These control signals command the switches 40 to operate in the first state (i.e., the A/P state) during a plurality of first times within a course of treatment, operate in the second state (i.e., the L/R state) during a plurality of second times within the course of treatment, and operate in the third state (i.e., the F/(A+L+R state)) during a plurality of third times within the course of treatment.

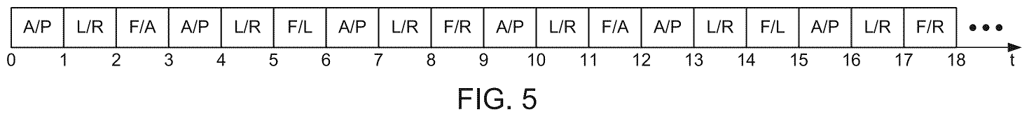

FIG. 5 depicts another example of a timing diagram for energizing the various sets of one or more electrode elements 20A, 20P, 20L, 20R, and 20F that will apply TTFields to the subject's brain in additional directions. During the first second of operation (i.e., between t=0 and t=1), an alternating voltage is applied between the anterior and posterior sets 20A, 20P, which will induce an electric field in the subject's brain with a general direction that runs from front to back. During the second of operation (i.e., between t=land t=2), an alternating voltage is applied between the left and right sets 20L, 20R, which will induce an electric field in the subject's brain with a general direction that runs from left to right. And during the third second of operation (i.e., between t=2 and t=3), an alternating voltage is applied between the fifth set of electrode elements 20F and the anterior set of electrode elements 20A, which will induce an electric field in the subject's brain with a generally vertical direction. The operation between t=3 and t=5 is identical to the operation between t=0 and t=2. During the next second of operation (i.e., between t=5 and t=6), an alternating voltage is applied between the fifth set of electrode elements 20F and the left set of electrode elements 20L, which will induce an electric field in the subject's brain with a generally vertical direction, but tilted upwards to the left. The operation between t=6 and t=8 is identical to the operation between t=0 and t=2. During the next second of operation (i.e., between t=8 and t=9), an alternating voltage is applied between the fifth set of electrode elements 20F and the right set of electrode elements 20R, which will induce an electric field in the subject's brain with a generally vertical direction, but tilted upwards to the right. This nine-part pattern then repeats (e.g., at least 100 times) for the duration of the course of treatment (i.e., from t=9 onward). Note that while each interval of time is 1 second long in the example above, shorter or longer durations (e.g., from 0.1 s to 100 s) can also be used. Note also that in the example above, the various timeslots are interleaved with each other and are mutually exclusive.

Once again, this approach applies TTFields to the subject's brain in additional directions without reducing the size of the transducer arrays that are positioned on the anterior, posterior, left, and right surfaces of the subject's head. And due to the directional nature of TTFields, adding the three new directions with vertical components to the two original generally horizontal directions can advantageously increase the overall effectiveness of the treatment. This approach also increases the overall volume that is covered by the TTFields (which can be advantageous when the body part being treated contains multiple regions of interest or a single region of interest that occupies a large volume within the subject's body), and/or to provide thermal management benefits (e.g., by reducing the duty cycle of transducer arrays that may be approaching their temperature limit, which can increase the amount of time that TTFields can be applied).

The hardware for implementing the FIG. 5 timing can be similar to the hardware described above in connection with FIG. 4, except that the bank of electronic switches is configured to route the output of the AC signal generator to either (1) appear across terminals A and P, (2) appear across terminals L and R, (3) appear across terminals F and A, (4) appear across terminals F and L, or (5) appear across terminals F and R, depending on the state of a control signal that arrives from the controller 50. In this embodiment, the controller 50 is programmed to generate the A/P, L/R, F/A, F/L, and F/R control signals illustrated in FIG. 5 in the illustrated sequence. These control signals command the switches 40 to operate in the first state (i.e., the A/P state) during a plurality of first times within a course of treatment, operate in the second state (i.e., the L/R state) during a plurality of second times within the course of treatment, or operate in one of three possible third states (i.e., the F/A state, the F/L state, or the F/R state) during a plurality of third times within the course of treatment.

Notably, in addition to the advantage provided by imposing TTFields in more directions, implementing the timing depicted in FIG. 5 provides another advantage with respect to the timing depicted in FIG. 3A. This is because in the FIG. 3A approach, the anterior set 20A is activated 66% of the time, which can cause the anterior set to heat up, which may necessitate a reduction in current to prevent that set from overheating. In contrast, when the FIG. 5 approach is used, the anterior, left, and right sets 20A, 20L, and 20R are each activated for just 44% of the time, which will spread out the heating more evenly between those sets, in which case overheating will become less of a concern.

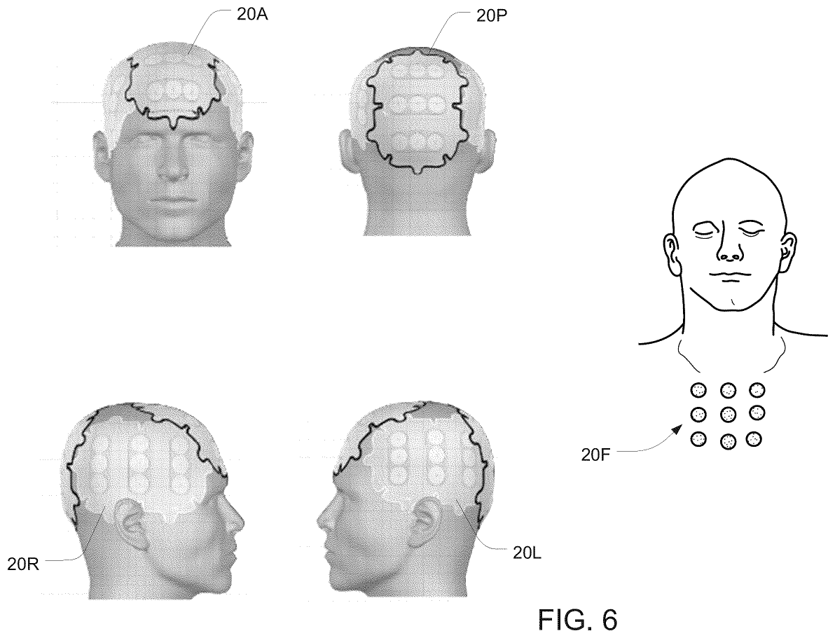

Note that the fifth set of one or more electrode elements does not have to be positioned on the subject's neck as depicted in FIG. 2. To the contrary, the fifth set could be positioned on the subject's torso (e.g., on the upper front portion of the subject's torso, as depicted in FIG. 6) instead of the positioning depicted in FIG. 2. When the fifth set is positioned on the subject's torso, the timing and the hardware are both similar to the situation described above in connection with FIG. 2-5. As yet another alternative, the fifth set can be distributed between two locations, with some of the electrode elements within the fifth set being positioned on the subject's neck, and the remainder of the electrode elements within the fifth set being positioned on the subject's torso. Here again, the timing and the hardware are both similar to the situation described above in connection with FIG. 2-5.

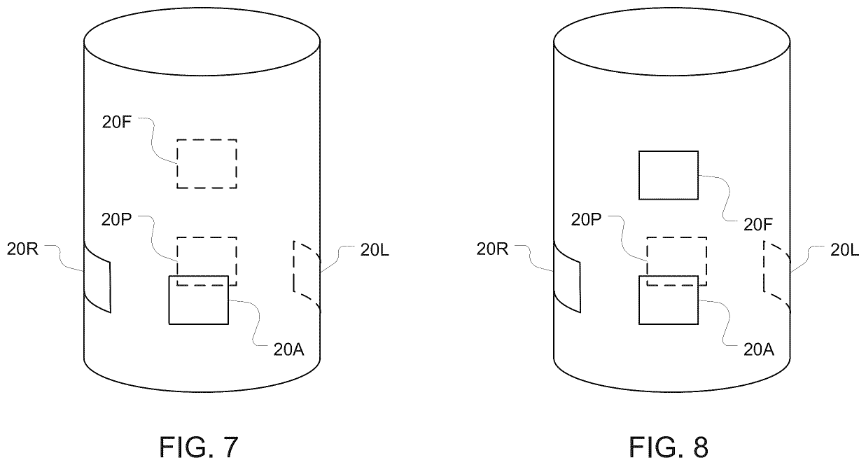

The approaches and embodiments described above in connection with FIGS. 2-6 are explained in the context of four sets of electrode elements positioned on the subject's head plus a fifth set of electrode elements positioned on the subject's neck or chest. These approaches and embodiments can be extended to a variety of other body parts including but not limited to torso, abdomen, leg, head, etc., as described below in connection with FIGS. 7 and 8.

More specifically, FIG. 7 depicts another approach for increasing the effectiveness of TTFields by increasing the number of directions at which the TTFields are applied. In this approach, a first set of one or more first electrode elements 20A is positioned on an anterior surface of the subject's body; a second set of one or more second electrode elements 20P is positioned on a first posterior surface of the subject's body; a third set of one or more third electrode elements 20L is positioned on a left surface of the subject's body; and a fourth set of one or more fourth electrode elements 20R is positioned on a right surface of the subject's body. In addition, a fifth set of one or more fifth electrode elements 20F is positioned on a second posterior surface of the subject's body (which is above the first posterior surface in the example depicted in FIG. 7, but could also be below the first posterior surface).

One timing sequence for energizing the various sets of one or more electrode elements 20A, 20P, 20L, 20R, and 20F in FIG. 7 is the three-part repeating pattern described above in connection with FIG. 3A (using similar durations of time). And the same hardware block diagram depicted in FIG. 4 can be used to apply the voltages to the various sets of one or more electrode elements 20A, 20P, 20L, 20R, and 20F when this timing sequence is used. When this approach is used, TTFields are applied to the body part in three directions instead of only two directions. In addition, this approach increases the overall volume that is covered by the TTFields (which can be advantageous when the body part being treated contains multiple regions of interest or a single region of interest that occupies a large volume within the subject's body).

Another timing sequence for energizing the various sets of one or more electrode elements 20A, 20P, 20L, 20R, and 20F in FIG. 7 is the three-part repeating pattern described above in connection with FIG. 3B (using similar durations of time). The same hardware block diagram depicted in FIG. 4 can be used to apply the voltages to the various sets of one or more electrode elements 20A, 20P, 20L, 20R, and 20F when this timing sequence is used. When this approach is used, TTFields are applied to the body part in three directions instead of only two directions. This approach also increases the overall volume that is covered by the TTFields (which can be advantageous when the body part being treated contains multiple regions of interest or a single region of interest that occupies a large volume within the subject's body). And furthermore, this approach can provide thermal management benefits (e.g., by reducing the duty cycle of transducer arrays that may be approaching their temperature limit, which can increase the amount of time that TTFields can be applied).

Yet another timing sequence for energizing the various sets of one or more electrode elements 20A, 20P, 20L, 20R, and 20F in FIG. 7 is the nine-part repeating pattern described above in connection with FIG. 5 (using similar durations of time). When this timing sequence is used, the same hardware block diagram depicted in FIG. 4 (with the modifications described above in connection with FIG. 5) is used to apply the voltages to the various sets of one or more electrode elements 20A, 20P, 20L, 20R, and 20F. The result is that TTFields are applied to the body part in many different directions instead of only two directions. This approach also increases the overall volume that is covered by the TTFields (which can be advantageous when the body part being treated contains multiple regions of interest or a single region of interest that occupies a large volume within the subject's body). And furthermore, this approach can provide thermal management benefits as described above in connection with FIG. 5.

Finally, the fifth set of one or more electrode elements 20F does not have to be positioned on the posterior of the subject's body as depicted in FIG. 7. To the contrary, the fifth set could be positioned on the anterior of the subject's body, as depicted in FIG. 8). When the fifth set of one or more electrode elements 20F is positioned on the anterior of the subject's body, the timing and the hardware are both similar to the situation described above in connection with FIG. 7, but in a mirror image. As yet another alternative, the fifth set can be distributed between two locations, with some of the electrode elements within the fifth set being positioned on the posterior of the subject's body, and the remainder of the electrode elements within the fifth set being positioned on the anterior of the subject's body. Here again, the timing and the hardware are both similar to the situation described above in connection with FIG. 7.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of using alternating electric fields to treat a tumor or prevent metastases in a subject's body, the method comprising:
positioning a first set of one or more first electrode elements on a first anterior surface of the subject's body;
positioning a second set of one or more second electrode elements on a first posterior surface of the subject's body;
positioning a third set of one or more third electrode elements on a left surface of the subject's body;
positioning a fourth set of one or more fourth electrode elements on a right surface of the subject's body;
positioning a fifth set of one or more fifth electrode elements on at least one of a second anterior surface of the subject's body and a second posterior surface of the subject's body;
applying an alternating voltage between the first set and the second set during a plurality of first times within a course of treatment;
applying an alternating voltage between the third set and the fourth set during a plurality of second times within the course of treatment; and
applying an alternating voltage between the fifth set and at least one of the first, second, third, and fourth sets during a plurality of third times within the course of treatment.

2. The method of claim 1, wherein the first times, the second times, and the third times are mutually exclusive, and
wherein the first times, the second times, and the third times are interleaved with each other.

3. The method of claim 1, wherein the first times, the second times, and the third times are mutually exclusive, and
wherein the first times, the second times, and the third times are interleaved with each other in a repeating pattern that repeats at least 100 times.

4. The method of claim 1, wherein the fifth set is positioned on the second posterior surface of the subject's body, and
wherein the step of applying the alternating voltage between the fifth set and at least one of the first, second, third, and fourth sets during the plurality of third times comprises applying the alternating voltage between the fifth set and the first set.

5. The method of claim 1, wherein the step of applying the alternating voltage between the fifth set and at least one of the first, second, third, and fourth sets during the plurality of third times comprises (a) applying the alternating voltage between the fifth set and the first set during some of the third times, (b) applying the alternating voltage between the fifth set and the third set during some of the third times, and (c) applying the alternating voltage between the fifth set and the fourth set during some of the third times.

6. The method of claim 5, wherein the fifth set is positioned on the second posterior surface of the subject's body.

7. A method of using alternating electric fields to treat a tumor or prevent metastases in a subject's body, the method comprising:
applying, during a plurality of first times within a course of treatment, an alternating voltage between a first set of one or more first electrode elements positioned on a first anterior surface of the subject's body and a second set of one or more second electrode elements positioned on a first posterior surface of the subject's body;
applying, during a plurality of second times within the course of treatment, an alternating voltage between a third set of one or more third electrode elements positioned on a left surface of the subject's body and a fourth set of one or more fourth electrode elements positioned on a right surface of the subject's body; and
applying, during a plurality of third times within the course of treatment, an alternating voltage between a fifth set of one or more fifth electrode elements positioned on at least one a second anterior surface of the subject's body and a second posterior surface of the subject's body and at least one of the first, second, third, and fourth sets.

8. The method of claim 7, further comprising:
positioning the first set on the first anterior surface;
positioning the second set on the first posterior surface;
positioning the third set on the left surface;
positioning the fourth set on the right surface; and
positioning the fifth set on at least one of the second anterior surface of the subject's body and the second posterior surface of the subject's body.

9. The method of claim 7, wherein the first times, the second times, and the third times are mutually exclusive, and
wherein the first times, the second times, and the third times are interleaved with each other.

10. The method of claim 7, wherein the first times, the second times, and the third times are mutually exclusive, and
wherein the first times, the second times, and the third times are interleaved with each other in a repeating pattern that repeats at least 100 times.

11. The method of claim 7, wherein the fifth set is positioned on the second posterior surface of the subject's body, and
wherein the step of applying the alternating voltage between the fifth set and at least one of the first, second, third, and fourth sets during the plurality of third times comprises applying the alternating voltage between the fifth set and the first set.

12. The method of claim 7, wherein the step of applying the alternating voltage between the fifth set and at least one of the first, second, third, and fourth sets during the plurality of third times comprises (a) applying the alternating voltage between the fifth set and the first set during some of the third times, (b) applying the alternating voltage between the fifth set and the third set during some of the third times, and (c) applying the alternating voltage between the fifth set and the fourth set during some of the third times.

13. The method of claim 12, wherein the fifth set is positioned on the second posterior surface of the subject's body.

14. The method of claim 12, wherein the fifth set is positioned on both a rear portion of the subject's neck and a front portion of the subject's torso.

15. An apparatus comprising:
a signal generator configured to generate an alternating output voltage;
a plurality of switches configured to, depending on a state of at least one control input, either (a) operate in a first state in which the alternating output voltage is routed to appear across a first output and a second output, (b) operate in a second state in which the alternating output voltage is routed to appear across a third output and a fourth output, or (c) operate in a third state in which the alternating output voltage is routed to appear across a fifth output and at least one of the first, second, third, and fourth outputs; and
a controller configured to send control signals to the at least one control input, wherein the control signals command the plurality of switches to operate in the first state during a plurality of first times within a course of treatment, operate in the second state during a plurality of second times within the course of treatment, and operate in the third state during a plurality of third times within the course of treatment.

16. The apparatus of claim 15, wherein the controller is configured such that the first times, the second times, and the third times are mutually exclusive, and such that the first times, the second times, and the third times are interleaved with each other.

17. The apparatus of claim 15, wherein the controller is configured such that the first times, the second times, and the third times are mutually exclusive, and such that the first times, the second times, and the third times are interleaved with each other in a repeating pattern that repeats at least 100 times.

18. The apparatus of claim 15, wherein the plurality of switches is configured to, while operating in the third state, route the alternating output voltage to appear across the fifth output and the first output.

19. The apparatus of claim 15, wherein the controller and the plurality of switches are configured such that:
during some of the third times, the plurality of switches routes the alternating output voltage to appear across the fifth output and the first output,
during some of the third times, the plurality of switches routes the alternating output voltage to appear across the fifth output and the third output, and
during some of the third times, the plurality of switches routes the alternating output voltage to appear across the fifth output and the fourth output.

20. The apparatus of claim 15, further comprising:
a first set of one or more first electrode elements positioned on a first anterior surface of a subject's body and wired to the first output;
a second set of one or more second electrode elements positioned on a first posterior surface of the subject's body and wired to the second output;
a third set of one or more third electrode elements positioned on a left surface of the subject's body and wired to the third output;
a fourth set of one or more fourth electrode elements positioned on a right surface of the subject's body and wired to the fourth output; and
a fifth set of one or more fifth electrode elements positioned on at least one of a second anterior surface of the subject's body and a second posterior surface of the subject's body, and wired to the fifth output.
